(19) United States
(12) Patent Application Publication
(10) Pub. No.: US 2008/0063697 A1
(43) Pub. Date: Mar. 13, 2008

(54) Use of Unactivated Calcium Exchanged Zeolites in Hemostatic Devices and Products

(76) Inventor: Robert L. Bedard, McHenry, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/530,339

(22) Filed: Sep. 8, 2006

(51) Int. Cl. A61L 15/00; A61K 33/12

(52) U.S. Cl. 424/445; 424/683; 442/123

(57) ABSTRACT

It is known that activated and partially activated zeolites are effective in hemostasis. However, the use of fully hydrated, unactivated zeolites has been ignored up to this point based upon a belief that it was necessary for such zeolites to concentrate certain components in the blood by removal of water. It has now been found that fully hydrated zeolites clot blood almost as quickly as fully activated zeolites that have been dehydrated without the potentially injurious exothermic response which may cause burns in tha case of fully activated zeolites.

### FIELD OF THE INVENTION

The present invention relates to blood clotting agents/medical devices and methods of controlling bleeding in animals and humans. More particularly, the present invention relates to zeolites that have a low heat of hydration.

### BACKGROUND OF THE INVENTION

Blood is a liquid tissue that includes red cells, white cells, corpuscles, and platelets dispersed in a liquid phase. The liquid phase is plasma, which includes acids, lipids, solubilized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances, animals, including humans, can be wounded. Often bleeding is associated with such wounds. In some instances, the wound and the bleeding are minor, and normal blood clotting functions without significant outside aid in stopping the bleeding. Unfortunately, in other circumstances, substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stanch the flow of blood in a timely manner. Moreover, severe wounds can be inflicted in very remote areas or in situations, such as on a battlefield, where adequate medical assistance is not immediately available. In these instances, it is important to stop bleeding, even in less severe wounds, long enough to allow the injured person or animal to receive medical attention.

In an effort to address the above-described problems, materials have been developed for controlling excessive bleeding in situations where conventional aid is unavailable or less than optimally effective. Although these materials have been shown to be somewhat successful, they are not effective enough for traumatic wounds and tend to be expensive. Furthermore, these materials are sometimes ineffective in all situations and can be difficult to apply as well as remove from a wound. Additionally, or alternatively, they can produce undesirable side effects.

Compositions for promoting the formation of clots in blood have also been developed. Such compositions generally comprise zeolites and binders. In a typical prior art zeolite composition, the water content is estimated to be about 1.54% or less. The water content is estimated by measuring the mass of material before and after heating at about 550° C. One attempt to deal with the heat of hydration problem was to provide a zeolite that has been rehydrated to a water content level of between 1.55 wt-% and 10 wt-% or dried to a water content level in that range.

The activated zeolite hemostatic material has been reported to cause superficial burns in some patients as a result of the large heat of hydration of the material that is exhibited when blood contacts the material. A product, has been described by Z-Medica, in their US 2005/0058721A1, that contains partially hydrated zeolite, which moderates the heat given off during use. The mechanism of action discussed in this application for coagulation enhancement involves partial blood dehydration and therefore concentration of clotting enzymes and cofactors by the zeolite. This operational hypothesis assumes that at least partial activation (dehydration) of the zeolite is necessary for effectiveness in the hemostat application. Surprisingly, it has now been found that such zeolites that are not even partially activated still promote a significant acceleration in the clotting function.

### SUMMARY OF THE INVENTION

Currently clay-bound calcium-exchanged zeolite A is being sold in an activated form as a hemostatic treatment for hemorrhages. The current market is primarily military, with substantial business being generated by the wars in Afghanistan and Iraq. Fully hydrated calcium-exchanged zeolites have been found to accelerate blood clotting substantially as effectively as partially or fully dehydrated forms of calcium-exchanged zeolites.

### DETAILED DESCRIPTION OF THE INVENTION

We carried out thromboelastographic (TEG®) analysis of the clotting time and clot strength of blood from several volunteers using both activated and fully hydrated Ca exchanged A zeolite and found that the clotting time [R(min). see following table] without zeolite were between 19.3 and 28.4 minutes, whereas the clotting time with varying amounts of fully dehydrated CaA were in the 0.8-2.2 minute range.

| | | | |
|---|---|---|---|
| Run # | Zeolite | Amount of Zeolite | R(min) |

Unactivated CaA

| | | | |
|---|---|---|---|
| 2 | Unactivated CaA zeolite | 5 mg | 2.9 |
| 2 | Unactivated CaA zeolite | 10 mg | 2.9 |
| 2 | Unactivated CaA zeolite | 50 mg | 3 |
| 5 | Unactivated CaA zeolite | 5 mg | 3.8 |
| 5 | Unactivated CaA zeolite | 10 mg | 3.2 |
| 5 | Unactivated CaA zeolite | 50 mg | 2.8 |

Control Runs

| | |
|---|---|
| 2 | 19.3 |
| 5 | 19.4 |

Activated CaA

| | | | |
|---|---|---|---|
| 5 | Activated CaA zeolite | 10 mg | 1.5 |
| 5 | Activated CaA zeolite | 10 mg | 1.5 |
| 5 | Activated CaA zeolite | 25 mg | 1.8 |
| 6 | Activated CaA zeolite | 5 mg | 2.2 |
| 6 | Activated CaA zeolite | 10 mg | 1.0 |
| 6 | Activated CaA zeolite | 30 mg | 0.8 |
| 6 | Activated CaA zeolite | 5 mg | 2.2 |
| 6 | Activated CaA zeolite | 10 mg | 1.9 |
| 6 | Activated CaA zeolite | 30 mg | 1.2 |

Control Runs

| | |
|---|---|
| 3 | 23.9 |
| 3 | 26.0 |
| 5 | 28.4 |
| 6 | 21.4 |
| 6 | 27.2 |

The clotting time for the fully hydrated zeolite was between 2.8 and 3.8 minutes. Although the time for the fully dehydrated zeolite was slightly shorter, the 2.8-3.8 minute clotting time for the hydrated zeolite is significantly shortened, without the exothermicity associated with the activated material. In fact, the shorter clotting time measured for the activated CaA is likely due to the higher temperature that the blood was heated to in those vials during the experiment.

The following protocol was used to test the blood samples.

The apparatus that was used was a TEG® analyzer from Haemoscope Corp. of Morton Grove, Ill. This apparatus measures the time until initial fibrin formation, the kinetics of the initial fibrin clot to reach maximum strength and the ultimate strength and stability of the fibrin clot and therefore its ability to do the work of hemostasis—to mechanically impede hemorrhage without permitting inappropriate thrombosis.

On unactivated samples:
i. Pipet 360 uL from red topped tube into cup, start TEG test
   On activated samples:
i. First, obtain the zeolite or other powder sample to be tested from lab. They should be weighed, bottled, oven activated (if needed), and capped prior to the start of the experiment. Zeolite samples are bottled in twice the amount that need to be tested. For example, if channel two is to test 5 mg of zeolite A and blood, the amount weighed out in the bottle for channel two will be 10 mg. For 10 mg samples, 20 mg is weighed out, etc. See note below for reason.
ii. For one activated run, 3 zeolite samples were tested at a time. An unactivated blood sample with no additive is run in the first channel. Channels 2, 3 and 4 are blood samples contacted with zeolite.
iii. Once ready to test, set one pipet to 720 uL and other pipet to 360 uL. Prepare three red capped tubes (plain polypropylene-lined tubes without added chemicals) to draw blood and prepare three red additional capped tubes to pour zeolite sample into.
iv. Draw blood from volunteer and bring back to TEG analyzer. Discard the first tube collected to minimize tissue factor contamination of blood samples. Blood samples were contacted with zeolite material and running in TEG machine prior to an elapsed time of 4-5 minutes from donor collection.
v. Open bottle 1 and pour zeolite into red capped tube.
vi. Immediately add 720 uL of blood to zeolite in tube.
vii. Invert 5 times.
viii. Pipet 360 uL of blood and zeolite mixture into cup.
ix. Start TEG test.

Note: The proportions are doubled for the initial mixing of blood and zeolite because some volume of blood is lost to the sides of the vials, and some samples absorb blood. Using double the volume ensures that there is at least 360 uL of blood to pipet into cup. The proportion of zeolite to blood that we are looking at is usually 5 mg/360 uL, 10 mg/360 uL, and 30 mg/360 uL

The R(min) reported in the Tables below is the time from the start of the experiment to the initial formation of the blood clot as reported by the TEG analyzer. The TEG® analyzer has a sample cup that oscillates back and forth constantly at a set speed through an arc of 4° 45′. Each rotation lasts ten seconds. A whole blood sample of 360 ul is placed into the cup, and a stationary pin attached to a torsion wire is immersed into the blood. When the first fibrin forms, it begins to bind the cup and pin, causing the pin to oscillate in phase with the clot. The acceleration of the movement of the pin is a function of the kinetics of clot development. The torque of the rotating cup is transmitted to the immersed pin only after fibrin-platelet bonding has linked the cup and pin together. The strength of these fibrin-platelet bonds affects the magnitude of the pin motion, such that strong clots move the pin directly in phase with the cup motion. Thus, the magnitude of the output is directly related to the strength of the formed clot. As the clot retracts or lyses, these bonds are broken and the transfer of cup motion is diminished. The rotation movement of the pin is converted by a mechanical-electrical transducer to an electrical signal which can be monitored by a computer.

The resulting hemostasis profile is a measure of the time it takes for the first fibrin strand to be formed, the kinetics of clot formation, the strength of the clot (in shear elasticity units of dyn/cm²) and dissolution of clot.

Fully hydrated zeolite powders have been found to be effective hemostats, thereby eliminating additional injury to trauma victims and patients due to burns caused by the heat of hydration upon application to wounds. These zeolite powders may be combined with a binder such as clay, alumina or silica. The zeolite powder that is functioning as a blood clot promoter may be contained within a porous carrier such as woven fibrous articles, non-woven fibrous articles, puffs, sponges and mixtures thereof. Fibers used to make such woven or non-woven fibrous articles may include aramids, acrylics, cellulose, polyester, chemically modified cellulose fibers and mixtures thereof These fully hydrated zeolite powders can be used as free flowing powders or incorporated into a bandage, gauze or other formed product for treatment of wounds. These blood clotting promoters have been found to increase the speed of clotting by a factor of between 2 and 12. Blood that was not treated with such blood clotting promoters exhibited clotting in about 20 minutes while the blood clotting promoters of the present invention reduced this time to less than 10 minutes and preferably to less than 5 minutes.

Various materials may be mixed with, associated with, or incorporated into the zeolites to maintain an antiseptic environment at the wound site or to provide functions that are supplemental to the clotting functions of the zeolites. Exemplary materials that can be used include, but are not limited to, pharmaceutically-active compositions such as antibiotics, antifungal agents, antimicrobial agents, anti-inflammatory agents, analgesics (e.g., cimetidine, chloropheniramine maleate, diphenhydramine hydrochloride, and promethazine hydrochloride), bacteriostatics, compounds containing silver ions, and the like. Other materials that can be incorporated to provide additional hemostatic functions include ascorbic acid, tranexamic acid, rutin, and thrombin. Botanical agents having desirable effects on the wound site may also be added.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A method for promoting blood clotting comprising contacting a blood clot promoter comprising fully hydrated zeolite with blood.

2. The method of claim 1 wherein said fully hydrated zeolite is ion exchanged.

3. The method of claim 2 wherein said ion is calcium.

4. The method of claim 1 wherein said blood clot promoter further comprises a binder.

5. The method of claim 4 wherein said binder comprises clay, silica or alumina or mixtures thereof.

6. The method of claim 1 wherein said blood clot promoter is contained within a porous carrier selected from the group consisting of woven fibrous articles, non-woven fibrous articles, puff, sponges and mixtures thereof.

7. The method of claim 6 wherein said porous carrier is a woven or non-woven fibrous article and the fiber is selected from the group consisting of aramids, acrylics, cellulose, polyester, chemically modified cellulose fibers and mixtures thereof.

8. The method of claim 1 wherein the blood which is clotted comprises blood flowing from a wound in an animal or a human.

9. The method of claim 1 further comprising the step of removing all or a portion of said fully hydrated zeolite from a wound.

10. The method of claim 1 wherein said fully hydrated zeolite is in the form of a free flowing powder.

11. The method of claim 1 wherein said fully hydrated zeolite is 10.01 to 25.0 wt-% water.

12. The method of claim 1 wherein said fully hydrated zeolite is 15.0 to 20.0 wt-% water.

13. The method of claim 1 wherein said fully hydrated zeolite promotes blood clotting at a rate about 2-12 times faster than in its absence.

14. The method of claim 1 wherein said fully hydrated zeolite promotes blood clotting in less than about 10 minutes.

15. The method of claim 1 wherein said fully hydrated zeolite promotes blood clotting in less than about 5 minutes.

16. The method of claim 1 wherein said blood clot promoter further comprises antibiotics, antifungal agents, antimicrobial agents, anti-inflammatory agents, analgesics, bacteriostatics, compounds containing silver ions or mixtures thereof.
